(19) United States
(12) Patent Application Publication
(10) Pub. No.: US 2022/0217130 A1
(43) Pub. Date: Jul. 7, 2022

(54) SYSTEM AND METHOD FOR A PATIENT INITIATED MEDICAL INTERVIEW USING A VOICE-BASED MEDICAL HISTORY QUESTIONNAIRE

(71) Applicant: Vancho Technical Sales, LLC, Dove Canyon, CA (US)

(72) Inventor: Vincent Michael Vancho, Dove Canyon, CA (US)

(21) Appl. No.: 17/701,804

(22) Filed: Mar. 23, 2022

Related U.S. Application Data
(63) Continuation of application No. 15/071,864, filed on Mar. 16, 2016, now abandoned.
(60) Provisional application No. 62/133,634, filed on Mar. 16, 2015.

(51) Int. Cl. H04L 9/40; G16H 10/60; G16H 10/20

(52) U.S. Cl. CPC H04L 63/083; G16H 10/60; G06F 21/6245; H04L 63/10; G16H 10/20

(57) ABSTRACT

A system and method for a patient to initiate and complete a voice-enabled, computerized, symptom-based medical history, at the time the patient is concerned about a medical problem. The interview mimics the real in-office medical interview between a patient and physician, complete with voice audible questions and responses, and is constructed to uncover factual information related to the patient's current complaint. Once the medical history is complete it is sent to the patients physician through the physician's Electronic Health Record system, the physician is then alerted that history report is waiting, and after reviewing the report, the physician calls the patient for a follow up interview to determine an appropriate next action for the patient.

### RELATED U.S. APPLICATION DATA

The present application is a continuation of and claims priority to U.S. patent application Ser. No. 15/071,864, filed Mar. 16, 2016, which claims priority from and the benefit of U.S. Provisional Patent Application No. 62/133,634, filed Mar. 16, 2015, the disclosure of which is hereby incorporated herein in its entirety.

### FEDERALLY SPONSORED RESEARCH

Not Applicable

### SEQUENCE LISTING OR PROGRAM

Not Applicable

### FIELD OF THE INVENTION

The present invention relates to a system and method to allow a patient to connect to his or her physician at the time the patient has a health concern. Using a voice-activated smartphone application the patient describes one or several chief complaints, and is then given a voice-driven, closed-question, computerized, interactive, branched-logic medical history interview questionnaire to gather all relevant background information related to the medical issue. Once the questionnaire is concluded, a report is generated, sent to the physician where it is reviewed, and a call to the patient is then made by the physician to have a follow up interview and determine next actions.

### BACKGROUND OF THE INVENTION

With the introduction of the Affordable Care Act (ACA) healthcare is undergoing significant changes. In order to cover all persons with some level of care, all persons are required to join a health plan. This will, in effect, cause the patient population to swell in coming years.

This is all happening while physicians are already pressed for time. A typical practicing physician will have between 1500 and 2500 patients, and sees patients all day in 20 minute examination appointment periods. Some examinations are for new medical issues, some are for follow-up appointments, while others are just concerns people have, and want to be checked out by their personal physician. Indeed, it is well known that being able to check on a problem in an early stage will tend to effect a positive outcome, as early symptoms can be warning signs for greater danger down the road. So when people learn to care about their health, they will, inevitably, want to talk more often with their physicians, and at an earlier stage when even slightest symptoms are presented.

While it might be an option for a physician to cut back on the number of patients, the practicality of today's healthcare situation dissuades this. There are simply not enough physicians to accommodate the growing, and aging, patient population. Even without the ACA, the Association of American Medical Colleges warns us that we face a shortfall of at least 130,000 doctors by 2025. Moreover, the United States already trails many other countries in the number of physicians per capita, at just 2.5 per 1,000 people. This is compared to nearly 4 per 1,000 in Germany and Switzerland.

To counteract this imbalance, the first call to arms should be to help each physician make his or her practice more efficient.

### SUMMARY OF THE INVENTION

The present invention relates to a system and method by which any patient can use a mobile computer device, such as a smartphone, to initiate and complete a voice-driven, interactive, logic-based, medical history questionnaire for any current medical complaint, then have the questionnaire's report automatically sent to the patient's physician. Upon receiving the report the physician would call the patient to confirm and enhance the history report, have a dialog with the patient, determine a course of next action, which could include doing nothing, or making an appointment with the physician's office, or sending the patient to a third party for further treatment. The system improves the continuity of care between physicians and their patients. The system can be used by the physician to minimize or eliminate trivial in-office visits while maximizing important in-office visits, thereby increasing practice efficiency, or to expand the practice to include more patients, or to develop supplemental patient services. Patients benefit by being empowered to communicate with their physicians as needed when a medical issue arises, and contribute to their care by providing relevant information allowing for quicker diagnoses, which could make a difference in their outcomes, while also eliminating travel burdens for needless office visits.

The medical history questionnaire mimics the in-office medical history interview in that it uses a branched logic similar to what a physician would use to conduct the typical medical interview, and covers all aspects of the medical history including: identifying information (gender, age); establishing the chief complaint; understanding the history of present illness; reviewing past medical, drug, family and social history; and completing a review of symptoms.

The history is conducted via a voice-generated application on a patient's smartphone or other remote communication device. Medical history questions are asked in a human voice, and may also be displayed on the smartphone screen such that the patient can confirm the question prior to answering. The patient can answer by voice, or use a graphical interactive interface on the smartphone to answer each question, or a combination of both. The questions can easily be repeated, and all are confirmed prior to commitment of the answer. Questions are essentially all of the closed type so that a required answer is expected to be a simple, single word.

The patient can use any voice interactive device, such as a smartphone, defined as a computer-processor enabled device, with an operating system such that it can make and receive phone calls and text-based messages; it can connect to the Internet, and may have third party applications that can be used on the device to collect clinical information. Other aspects of the present invention can have the patient using a smart watch, a cellular phone, a standard wired, landline telephone, or a desktop or laptop computer with a microphone and speaker and capable of communicating through voice over the Internet.

In another aspect of the invention the medical history questionnaire is conducted via an interactive, synthesized human voice, but the return answer can be by text by touching an icon of the expected answer. For example, instead of saying “1” the user can touch the number “1” and the answer is the same.

In another aspect of the invention the ability of the physician to contact the patient can be enhanced by adding video to the voice call.

The report output is sent to the physician's EHR system to be included into the patient record. The report can also be sent to the patient's Personal Health Record (PHR), which is usually part of a third party system for patients to keep their own health records. In another embodiment the report can be sent directly to the physician.

Once the medical history report is received by the physician, the physician can scan the medical history, and check any other patient information on record, if desired, prior to making direct contact with the patient. Direct contact is by voice and established by a direct phone call between the parties. In other aspects of the invention, however, the voice call can be made over a cellular network between cell phones or smartphones or smart watches, or it can be made via a wired or wireless internet call on a computer. Additional aspects include video communication if it can be supplied concurrently with the voice communication. Video can aid in establishing nonverbal communication cues that might ordinarily be picked up in a face-to-face medical interview.

The medical history report, according to the present invention, should be formatted such that it presents all relevant factual information in the easiest possible manner for a physician to scan the document in 1-2 minutes prior to calling the patient. Armed with relevant medical history information the physician can, while having direct voice contact with the patient, ask open ended questions to establish or confirm a diagnosis of the patient's problem in a short period of time, after which a decision on the best next course of action can be made by the physician and agreed to by the patient.

The present invention allows a patient to initiate and complete an easy-to-use medical history questionnaire that substantially reduces the total amount of direct physician-patient encounter time while it provides essentially all of the same constituents of an in-office encounter and a high level of continuity of care.

### BRIEF DESCRIPTION OF THE DRAWINGS

FIG. 1 shows an overview of the process methodology.

FIG. 2 shows the communication gateways for sections of the process methodology.

FIG. 3 is a representation of the system elements associated with administering a voice-based questionnaire.

FIG. 4 shows the difference between open and closed ended questions in a medical history.

FIGS. 5A-B shows a graphical input smartphone screen to provide the chief complaint.

FIGS. 6A-B show how a dosed type of medical question is presented on a smartphone.

FIGS. 7A-C illustrate how a lengthy closed type of medical question is presented on a smartphone.

FIG. 8 shows devices used during the physician-patient encounter.

FIG. 9 illustrates the amount of time reduction that is possible using the present invention.

### DESCRIPTION OF THE PREFERRED EMBODIMENT

Embodiments of the present invention will be described with the accompaniment of the drawings attached within this disclosure. Additional embodiments will be described herein such that those skilled in the art will fully understand the scope of the present invention. Within this invention disclosure the term “physician” is used to describe a person who communicates with a patient of record. Generally, in primary healthcare that person is a medical doctor, family physician or internist. However, people skilled in the healthcare field recognize that jurisdiction of a person's health does not begin and end with a physician, that many other individuals supporting the physician can be involved. As such, the term “physician” in this disclosure can mean any person or entity for whom the patient's physician has given such responsibility to act as a caregiver for the patient on behalf of the physician. This can mean, nurses, physician assistants, or other qualified clinical personnel who may work for the physician, or be contracted as a third party by the physician. Also, in this disclosure, the term “application” and “app” are used interchangeably and refer to a computer program that works in conjunction with a processor.

While the aging population is expanding, it does so armed with an increasing understanding of new and available computer and communication technologies that could provide at least a partial solution to the problem. By having a capability to easily connect and communicate with their patients before patients choose to make appointments, physicians could eliminate needless in-office encounters that could very easily be managed remotely with much less time and effort.

However, the quality of the system that is put in place to manage the remote, non-office-based interaction and communication with patients is critical to provide the highest level of efficiency, which could be defined as optimizing each in-office visit by treating patients remotely, at less time and cost than an in-office visit, with a system and method that provides a comparable experience to an in-office visit, until such time that the physician absolutely needs to see the patient. A system such as this will save time and money for both the physician and patient.

A typical office examination visit lasts about 20 minutes. During this time the physician must conduct a medical history followed by a physical examination, order any laboratory work, and hypothesize a diagnosis. Several studies published in the National Center for Biotechnology Information (part of the National Institute of Health) support the concept that most diagnoses (˜80%) are made from the medical history versus the physical examination (˜10%) or lab investigations (˜10%). An article published in the Harvard Business Review (Web Edition, Jan. 28, 2014, Gregory Sorensen, CEO, Siemens Healthcare) cites that in the first 15 minutes of a patient encounter up to half of all medical costs are set in motion.

Therefore, the medical history, it can be argued, is of high importance in not only supporting a diagnosis, but in having a significant effect on a majority of medical costs relating to the patient's illness.

The medical history has several parts: Identifying the patient (gender, age); establishing the chief complaint; understanding the history of present illness; reviewing past medical, drug, family and social history; and completing a review of symptoms. Not all of these are taken in equivalent measures in every interview. For example, in a patient that is being seen for the same issue but at different times, the physician would take an “interval history” which would not have to restate past medical history that has already been previously given.

According to medical literature, the in-office medical history is comprised of two distinct areas for patient reporting: what the patient says, and how it is said. What the patient says is a function of what the physician asks and requires verbal communication. It is factual information about the issue at hand, and it depends totally on the physician's ability to be in the moment, to ask relevant questions, and to be as thorough as necessary to capture all important information.

In a medical history determining what the patient says requires questions that are structured to be both open and closed with respect to their responses. Closed questions allow answers that are discrete and quantifiable, requiring answers such as “yes” or “no” to the physician's questions. An example might be where the physician asks the patient, “Was your father ever diagnosed with diabetes?” They are best used when a specific area of information is examined. Closed questions provide most of the objective factual data of a history. Open questions, on the other hand, allow for more verbal communication and are intended to elicit more subjective data from the patient. The most obvious open question usually happens at the beginning of a history interview with the question like, “How are you feeling today?”

How the patient says something provides insight into the patient's frame of mind, and is a function of both nonverbal communication and verbal communication. Through the patient's behavior during the interview (e.g., voice tone and quality, facial expressions, posture, gestures) he or she communicates emotional concerns, reactions to illness, and style of relating to others. Sudden shifts of topic, avoidance of certain issues, and the flow of spontaneous associations may point to concerns that cannot be found by analyzing the patient's words alone.

In a typical medical history interview the majority of time spent by the physician is used to gather specific quantified data through closed questions, which, as previously stated, is done exclusively through verbal communication without any prejudice to how the words are stated. By allowing this portion of the medical history to be automated, without the intervention of the physician, the physician frees his time and improves his or her efficiency. The key is, however, that the automated medical history interview must be as close as possible to a real physician encounter, which is conducted through a series of interactive voiced questions from the physician to the patient that can change direction based on previous questions. Therefore, ideally, a type of automated voice-enabled, branched-logic based interactive interview questionnaire must be used.

Moreover, for the patient, the situation should be one in which the patient initiates the automated medical history interview, at the time when the patient is having an issue. Symptoms which appear at a particular time may quickly go away, and the patient may not be able to remember the quality of specific symptoms if so much time is taken between the onset of symptoms and when the patient finally gets in to see a physician in an office visit. The patient should be able to initiate the automated questionnaire from any location, at any time, and would be able to cover any type of concern the patient is having for his or health at the moment. Additionally, once the patient completes this automated medical history questionnaire, a simplified report should be created for the physician to read or hear, followed by a direct communication between the physician and patient, whereby the physician can cover more subjective questions to glean any information about the patient's wellbeing.

Current prior art does include the ability of a patient to initiate remote forms of gathering oblique medical information about a patient, that is delivered to a physician. However, all the current applications of prior art are text based, and require picking from a preselected form a set of symptoms, and do not replicate the in-office interview process. For example, prior art defined by Kalamas in U.S. Pat. No. 8,571,890 teaches how to generate a medical history based on a patient's medication list. In prior art by Hudson in U.S. Pat. No. 8,700,424 the patient must use a limited symptom drop down menu search as a way to gather the medical history. Additionally, U.S. Pat. No. 8,423,387 by Mirza allows the patient to input a complaint, and then looks up possible symptoms to choose from, with no follow up computerized, logic-based interrogative questioning similar to what is done by a physician in a real office visit. An example of published articles that evaluated computer-based medical histories taken by patients at home is in the Journal of American Medical Informatics Association, July-August 2012, in which a preselected series of questions were administered to people at home. None of these teachings exemplify what a real world medical history interview is like, whereby the physician will ask a question, and depending on the answer, will probe another area of concern in a branched-logic process until all possible data gathering is complete.

In the current prior art there is also at least one automated medical history questionnaire that can be used to collect symptom based medical history information, but it is always initiated and administered by the physician once the patient already establishes an appointment, and it is text based. The questionnaire is given when the patient enters the office setting, and requires a computer to visually provide the text based question and text based response. If the questionnaire is set to run remotely, such as at home prior to the office visit, initiating the questionnaire process is so complex that it must be set up by someone at the physician's office, thereby offsetting the improvement in efficiency by the physician's office as a whole. Finally, the questionnaire requires a computer with sufficient screen size to produce extended text based questions, which may have multiple choice answers. So running the program on a mobile device such as a smartphone or smart watch is difficult if not impossible.

So it stands to reason that when communicating with a patient remotely, to be able to effectively generate a good patient medical history, the process should be as close as possible to a live, interactive, face-to-face medical history conversation between the patient and physician. In this regard a medical history administered remotely should include the following: (1) physician questions should be capable to run via voice to be easily understandable, and not confined to written text-based questions; (2) close-type questions should be used so that answers would be simple “yes” or “no” or “I don't know” or multiple choice based. This would allow the patient to make sure answers are clear and correct; (3) The history should capture quantifiable information similarly to that which could be captured during an in-office examination, including: Identifying patent information (gender, age); establishing the chief complaint; understanding the history of present illness; reviewing past medical, drug, family and social history; and completing a review of symptoms; (4) a written or voice enabled report of the medical history should be generated, which is added to the patient's Electronic Health Record (EHR) in the physician's EHR database for complete documentation purposes, with the report formatted for maximum ease of use by the physician; (5) an ability for the physician to quickly follow up directly with the patient, via voice communication and, if possible, to include video, to confirm the written history profile, ask open ended questions to elicit additional qualitative information from the patient, perhaps even monitor patient health data in real time; and finally to discuss any follow up necessary to effect a best possible next action, which could include a request for the patient to come in for an appointment, or to send the patient to a healthcare facility immediately for further help, or to simply do nothing and watch for additional symptoms to occur.

What is needed is a system and method for a physician to allow his or her patients to be able to initiate a simple, automated, voice-driven medical history questionnaire similar to an in-office, face-to-face medical history, in any location and at any time the patient feels threatened by an emerging medical issue. The automated questionnaire will eliminate the need for a physician's presence for the medical interview, yet will provide the necessary background medical history information for a current medical complaint. The physician should receive a simply formatted report with the entire current complaint-related medical history immediately after the completion of the questionnaire, and have the ability to contact the patient by voice, inclusive or not with video, and have a consultation to determine the next course of action for the patient.

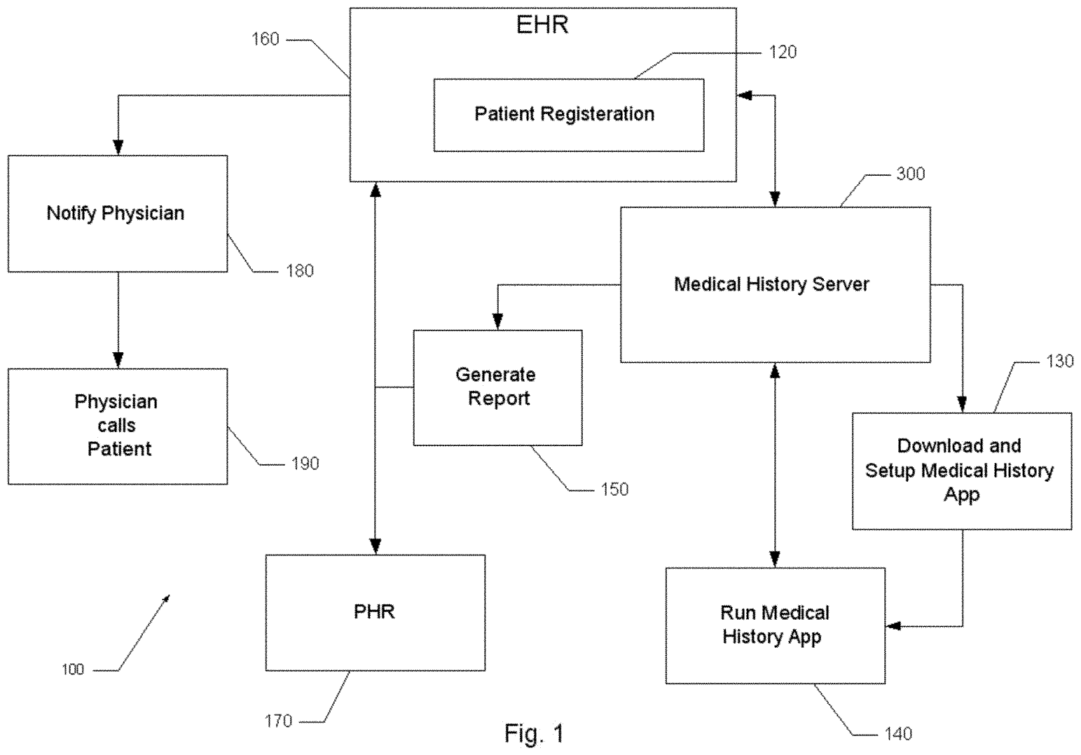

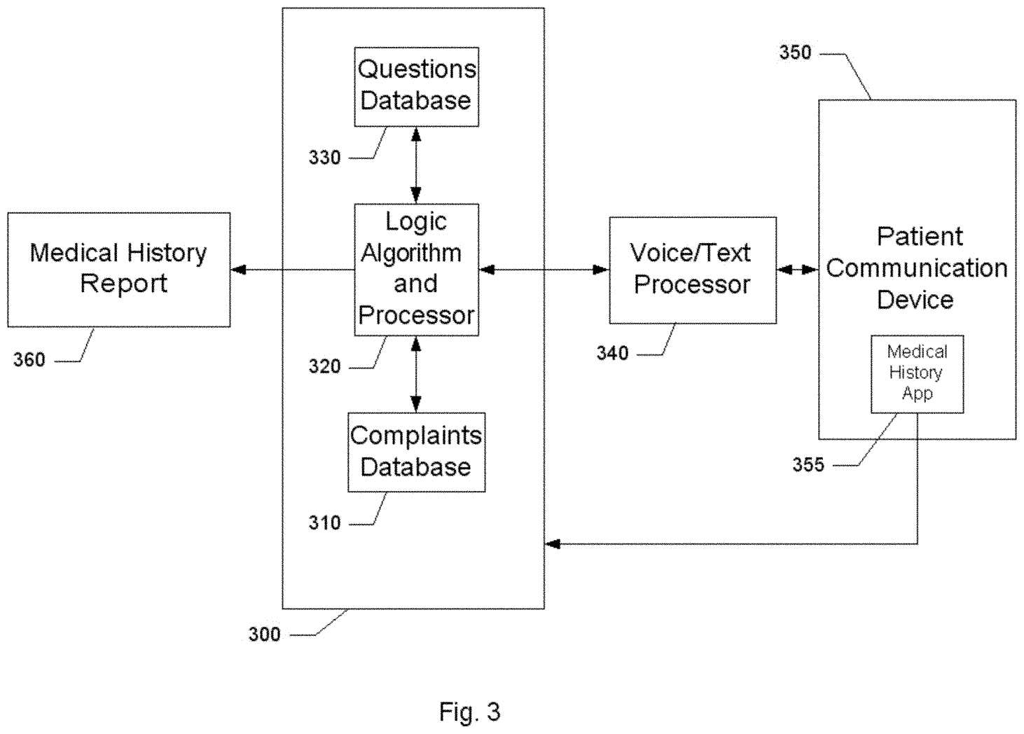

FIG. 1 is a block diagram of the overall process methodology 100 of the present invention. It begins with a physician who would be required to have an EHR 160 for the practice, which is in accordance with current requirements of the Affordable Care Act. Patient registration 120 would occur within the EHR, and would include a co-registration with a medical history server 300, discussed in more detail in FIG. 3. Once registered, data links would be established between the EHR 160 and medical history server 300 using compliant procedures, such as communication protocols suggested by the Health Insurance Portability and Accountability Act (HIPAA) and the ACA.

The physician would designate within the EHR all patients who should have access to the medical history server 300. Once so designated, the physician's office would notify all designated patients and invite them to register for the service. Patient registration 120 would include the transfer of any patient identifiers into the medical history server 300 necessary to run the system, for example the patient's gender and date of birth and a patient ID number held in the EHR 160. Patient registration 120 would also include a second opt-in agreement to allow the physician to have access to the patient's PHR. Once documented and registered, the patient would be given access to a medical history application download 130 which will be used to run the actual medical history program. In the preferred embodiment of the methodology, the application would be a smartphone application, able to be loaded on a smartphone and used in any remote setting to take the medical history. However, the medical history application can be a programmed application that runs on the patient's remote device, which may be a mobile device such as a smart watch, PC tablet, or laptop computer, or it may be a fixed device such as a desktop computer.

Once the patient downloads the medical history app on his or her smartphone the patient is now able to run the medical history app 140 to take a voice responsive medical history from the medical history server 300 whenever there is a concern that the patient has an encroaching medical issue. After the medical history is complete the medical history server generates a medical history report 150 which is sent to the physician's EHR 160. Once received by the EHR a physician notification 180 is sent by the EHR to alert the physician that a medical history report is ready. At this point the physician can access the EHR to review the text based report, and while in the EHR the physician can also check any part of the patient's record to begin to set some early hypothesis of the patient's issue. Simultaneously, while the report is sent to the physician's EHR 160, a similar report is sent to the patient's PHR 170, should the patient have agreed to have a copy sent to the patient's PHR during patient registration 120. The PHR is a collection of the patient's medical information that is controlled by the patient. While patients may end up over time having several doctors, the patient will generally have only one PHR. By sending the medical history to the patient's PHR, a continuity of record is established for the patient. Returning to FIG. 1, the physician finally calls the patient 190 and discusses the current complaints and issues of the patient, asking all the necessary questions, open or closed type, to get a more accurate diagnosis.

While the aforementioned description is one embodiment of the method for the present invention, there are a number of alternative embodiments. Although physicians are quickly establishing the use of EHR systems, it is not a requirement of the medical profession; therefore, an alternative embodiment would be to have the physician register directly into the medical history server 300, where he could then add his patients who should have access to the medical history. In this case patients would also have to register within the medical history server 300 to download the required smartphone medical history questionnaire application.

Downloading and setting up the medical history app would be done directly on the smartphone, and running the medical history app would be predominantly via voice, but could be supplemented by text or interactive touch, depending on the smartphone device.

An alternative embodiment of the preferred system is that, rather than run the medical history app on a smartphone, the patient can use a PC tablet or laptop computer or computer with voice enablement, or a standard telephone or cellular phone. In the case of using a telephone or cellular phone, neither of which have computer processing capability, the medical history app would reside within or in conjunction with the medical history server, and operate exclusively on a voice responsive arrangement. In another alternative embodiment of the system, the patient can use a smart watch, which is a wearable device with microprocessors that are typically in communication to a smartphone which is connected to the Internet, but in separate embodiment the smart watch could connect directly to the Internet.

Notification of the physician 180 may be done by a phone call to the physician's smartphone, but could also be done in alternative embodiments by a text sent to the physician's smartphone or a voice call via a PC tablet or computer or a standard land line phone call to the physician.

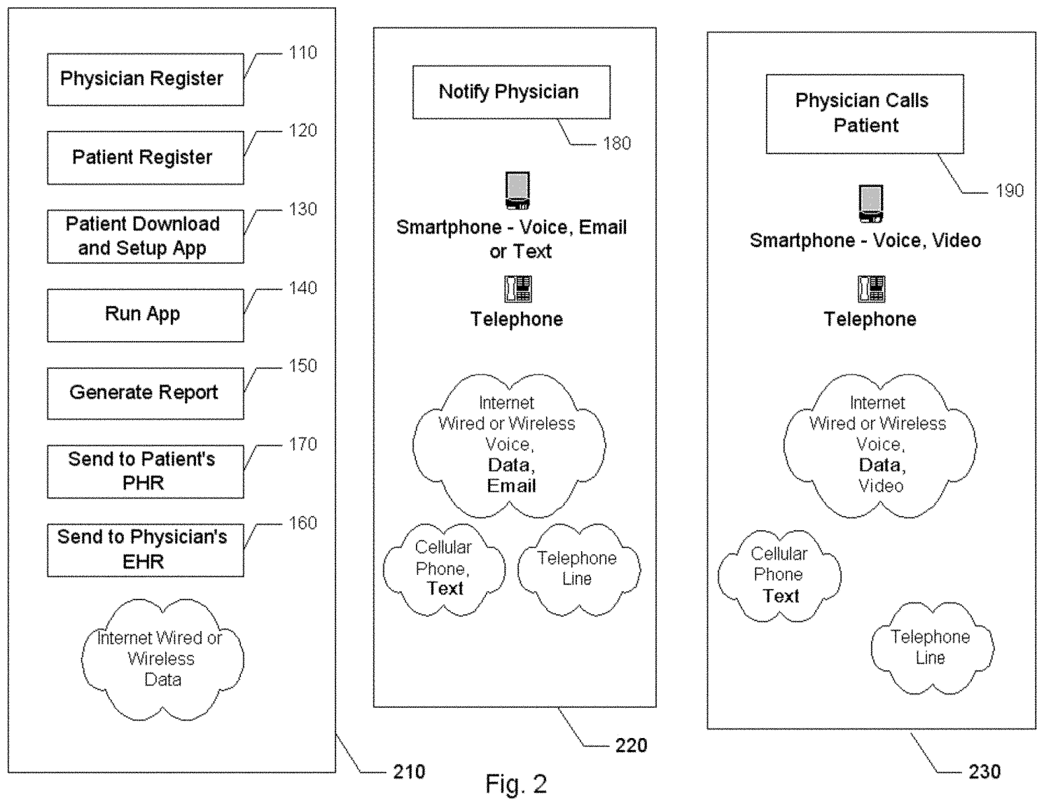

The nature of the process of the current invention requires communication between the physician and patient, and between database systems and processors located on network servers. FIG. 2 is a review of the embodiments of various communications. Processes that require data communication via the Internet are located in area 210, and include the patient's registration into the system 120, which, in the preferred embodiment would be to register in the physician's EHR, and done via an Internet interface. The patient downloads the App from the medical history server 300 via a network connection, and runs it over the wired or wireless Internet connection 140. Because the medical history server generates the report 150, it is sent via an Internet interface to the physician's EHR 160 and the patient's PHR 170. The term “Internet” in regards to the present invention, and as stated above, includes, but is not limited to, wired Internet services provided by cable and satellite companies, wireless internet services provided by cellular communication companies, or any other form of Internet communication that those skilled in the art of network communication infrastructure would understand as conventional means of data communication.

To notify the physician that a medical history is taken area 220 shows that the notification could be done by voice using the smartphone as the preferred embodiment, but in alternative embodiments an Internet voice call could be done using the PC tablet, laptop computer or desktop computer, or a direct call with a standard landline voice phone or cellular wireless phone, or a text message could be sent using cellular or Internet smartphone technology.

Continuing on FIG. 2, area 230 shows that after the physician receives notification the medical history report is ready in the EHR, and completes a review of the patient's medical history and EHR data, in one embodiment, the physician would call the patient to have a direct person-to-person voice-based follow up medical interview. In some embodiments the voice call would be made from the physician to the patient on the patient's smartphone. However, on certain smartphones, which have a video camera there exists a capability to have a corresponding video connection with the voice connection, and in these instances, the physician could elect to have a face to face medical interview. In an alternative embodiment, the physician could call using a tablet PC, laptop computer or desktop computer that is capable of voice communication, and could include video should both the patient device and physician device be capable of transmitting video between them. Another alternative embodiment would be to have a simple land based telephone call.

Turning now to the system involved in executing the medical history questionnaire, FIG. 3 shows the elements needed for a patient to initiate and complete the automated voice-driven medical history questionnaire on a patient communication device 350 using an embedded medical history app 355. In the preferred embodiment, the patient communication device 350 is a smartphone and the medical history app 355 is a smartphone app. The medical history questionnaire runs from a medical history server 300 and includes a complaints database 310 and a database of questions 330. A computer processor embedded with logic instructions 320 analyzes the incoming complaints, the outgoing flow of questions, and proper recording of an answer for each question. Additionally, a voice/text processor 340 located as part of, or separately from, the medical history server, and serially located between the medical history server 300 and the patient's smartphone 355, exists to translate outgoing text into voice recognizable language, and return voice recognizable language into specific text instructions. Once the final question is asked, the processor 320 compiles all answers into a formatted medical history report 360 and sends the report to the physician's EHR.

The detailed system and process is as follows: After the physician designates the patient as a candidate for using the method and system of the present invention, the patient can begin his or her registration process. During patient registration (FIG. 1. 120), the patient provides information into the physician's EHR (FIG. 1, 160) that also uniquely registers the patient for use of the medical history server (FIG. 1, 300). Subsequent to that, the EHR establishes credentials within the medical history server 300 that uniquely recognizes the patient, provides the patient access to the medical history server, and associates the patient to the physician's EHR, and, optionally, to the patient's PHR. Once all credentials are confirmed and executed, the patient is then instructed to download and install a medical history app from the medical history server 300 capable of running on his or her communication device 350, which, as previously stated, is a smartphone. The actual smartphone app 355 sets up necessary secure communications between the smartphone and medical history server 300, and confirms registration credentials with the medical history server to take a medical history.

At the time a patient has an occurrence of a medical incident, the patient uses the smartphone 350 to start the smartphone App 355 and initiate a medical history questionnaire (FIG. 1, 140, the process of running the app). Because the smartphone and app have already been credentialed with the medical history server the software simply begins to run. The very first question provided by the smartphone app 355 is an open ended interrogative that requests how the patient is feeling at the moment. The answer to the open ended question is dissected to provide necessary starting points for the branched medical logic questionnaire used during the medical history interview. The patient's voiced response is converted to text by the voice/text processor 340 and the resulting text is analyzed by the logic processor 320 and key words and phrases are extracted to match one or more complaints existing in a complaints database 310.

Each complaint is associated with a set of starting questions, much like a physician would use, to probe for particular symptoms that might set about a pathway to a diagnostic condition. The questions are located in a questions database 330, and are for the most part, closed ended questions requiring a simple single word answer, even though the question itself might be complex in nature. This allows the medical history to probe deeply, and accurately, into the patient's history, and minimizes the influence of patients with different dialects, use of slang, and strong accents.

Overseeing this entire process is a logic algorithm and associated processing engine 320 which analyzes patient's answers through the voice/text processor 340, and changes the questioning tactics as needed to establish whether a diagnostic pathway is credible or not. This means, for example, that a patient might first enter the medical questionnaire with a complaint of “chest pain” and after a thorough interrogation of heart history issues or symptoms for acute heart problems, the logic processor 320 may find a more credible pathway when exploring questions related to food problems and gastric disorders, which would lead to examination of intestinal issues.

All information that is passed from the logic processor 320 to the patient's smartphone 350 passes through the voice/text processor 340. This processor essentially translates text based information to speech and voice based information to text. Text generated by the logic processor 320 is presented on the smartphone as audible synthesized speech, and includes all questions that are asked during the medical history interview. Returning voice responses are also converted to text by the voice/text processor 340 for processing by the logic processor 320. The voice/text processor 340 can be part of the medical history server 300, or an independent processor, or a processor that is part of the smartphone 350. In the preferred embodiment, the total functionality of the voice/text processor 340 is shared among all three devices.

The medical history server 300, is an computer server attached to the Internet, and can be an stand-alone PC comprising both the questions database 330 and complaints database 310 together along with the logic algorithm and processor 320 in one configuration, or each of the elements can exist separately among many Internet servers, or the system can be implemented in a “cloud” server arrangement, in which all three devices are administered entirely over the Internet using virtual servers and multiple backup arrangements.

In alternative embodiments, the patient communication device 350 could be a smart watch, defined as a mobile wearable watch with a microprocessor that communicates to the smartphone via a wireless technology. In this embodiment, the smartphone and the smart watch would have a common embedded application that enable incoming voice instructions on the smartphone to be transferred to the smart watch where it can be audibly heard by the patient. The patient could then provide an voice audible answer into the smart watch where it is received and passed to the smartphone, which sends it to the medical history server 300. In another embodiment the smart watch could be directly connected to the Internet and with proper programming would be enabled to initiate the medical history questionnaire directly with the medical history server 300.

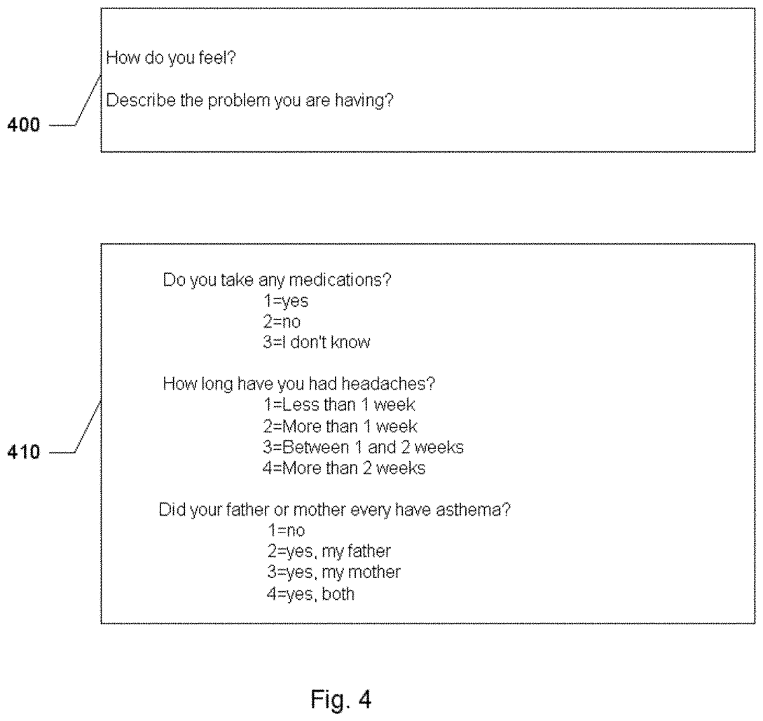

With the exception of the initial complaint request. database questions used by the logic processor 320 are generally of the closed end type, and in the preferred embodiment of the invention more than 75% of the questions represented in the questions database 330 are comprised of the closed type. FIG. 4 shows the difference between the initial open ended request 400 and questions of the closed end type 410. The answer to the open ended question 400 could be of any length. In actual practice, people familiar with the art of voice enabled application would realize that even this single entry question could not allow an unlimited return answer. As such, if all questions were of this type, the entire questionnaire might result in a level of complexity and time to make it cumbersome. Therefore, by having the medical history questionnaire limited with respect to open ended questions, simplicity is maintained. Closed ended questions 410 result in answers that are discrete, even though the questions themselves may be long and complex, the answer is always reduced to a discrete word, and as such, the resulting information is accurate and quantifiable. For example, the question may request the patient to: “Say 1, if you have had headaches for less than one week; Say 2 if you have had headaches for more than one week; Say 3 if you have had headaches for between 1 and 2 weeks; Say 4 if you have had headaches for more than two weeks.”

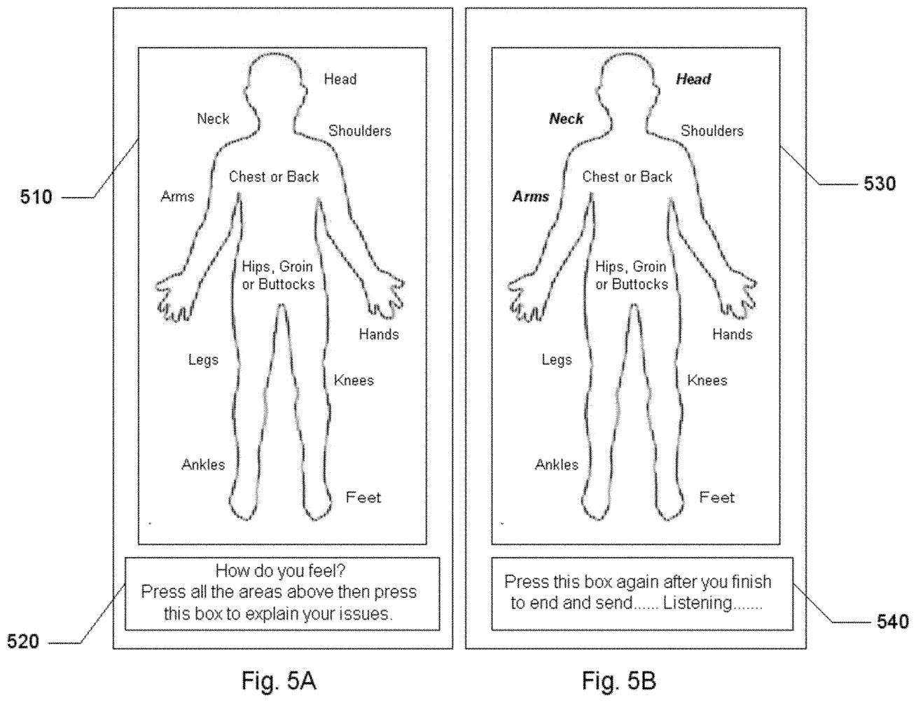

A preferred embodiment of the initial “how are you feeling” question presented on the patient's smartphone 350 is shown in FIGS. 5A and 5B. FIG. 5A represents the displayed image of the smartphone after starting the medical history, and consists of a graphical representation of the body in the top portion 510, and a text area at the bottom with instructions to the patient 520. In order to understand the free speech of a patient with a chief complaint, being able to locate the major area afflicted is significantly helpful in reducing process time of the logic processor (FIG. 3, 320). FIG. 5B shows how the display changes after performing the instructions 520 in FIG. 5A. The body graphics 530 have changed to reflect those areas which have been touched by the patient, and the text area 540 has changed into a listening mode with an action to press the area 540 when finishing the list of complaints associated with the graphical areas represented. At this point the patient's voice recording of his complaints is converted to text by the voice text processor 340, and all the text information is sent to the logic processor 320 to analyze the content and extract the correct starting questions for the medical history.

In an alternative embodiment the displayed area 520 can be used to type in a complaint, if the patient is in a location where it may be distressing to voice the complaint while others are around. An example may be that the patient is in a public place with other people present, and it would be embarrassing to speak into a voice-only system so that other persons can hear the medical complaint. In this embodiment, the area 520 would provide the patient with instructions to choose between texted or voiced instructions, or could even be made to provide a path to both, just to make sure the correct complaint was presented to start the questionnaire.

In yet another alternative embodiment. FIGS. 5A and 5B could be done entirely in a voiced condition, if, for example, the patient is using a smart watch with direct connection to the Internet. In this case the medical history application would ask the patient to speak some key words about the complaint, such as “heartburn, pain in left abdomen, headache, etc.” Responses would be collected and converted to text and specific key words would be parsed out as starting conditions.

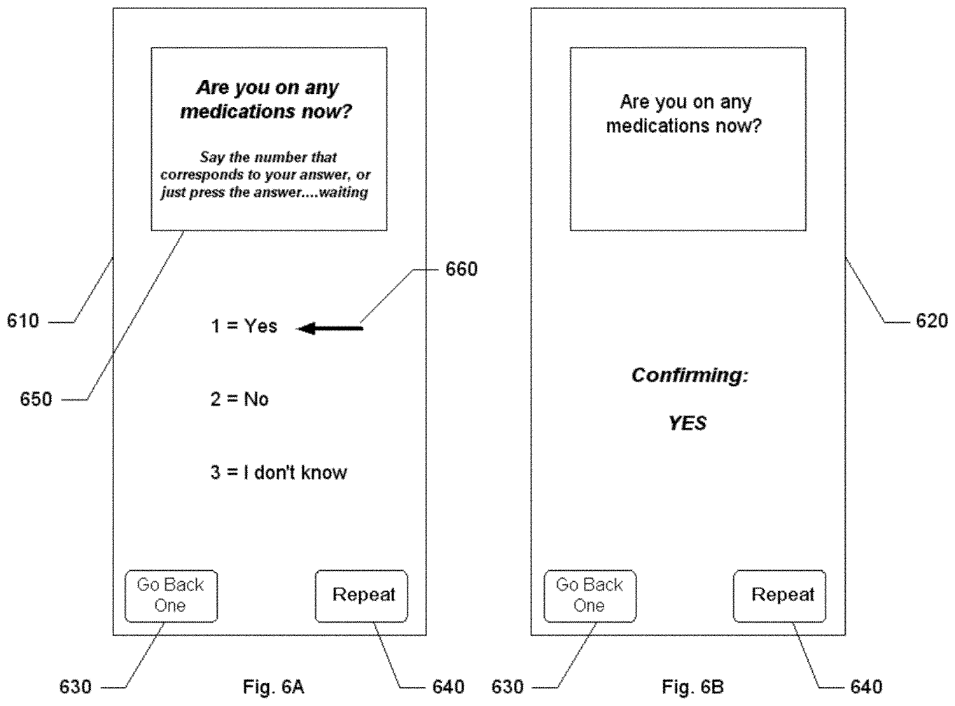

Once the initial complaints are resolved, the logic processor 320 in the medical history server 300 begins to send closed ended questions through the voice/text processor 340 to be presented on the patients smartphone 350. FIGS. 6A and 6B show the smartphone display with a closed end question presented. In area 610 of FIG. 6A a question is shown at the top section 650, and, while text is displayed, the voice repeats the same question and provides instructions on what to do next. Once the instructions are said the smartphone stands ready for a voice response, and the area 650 adds the last word “waiting,” which indicates the smartphone is waiting for the patient to select from the set of possible answers. The patient can respond by either saying the number associated with the answer, or if the smartphone is touch enabled, just touch the answer. This provides both an audio and visual mechanism to make sure the question is fully understood. (The arrow 660 is not part of the display, and is for illustrative purposes only to indicate that the patient responded “yes.”) Display 610 in FIG. 6A changes to display 620 in FIG. 6B to display the question, “Are you on medications now?” The answer is confirmed from the logic processor 320 through the voice/text processor 340 to the smartphone 350 with the audible words: “Confirming . . . yes.” When the answer is voiced, there is a timer setting which, when the timer end is reached, the program will automatically continue to the next question. in an alternative embodiment, a message on the display could instruct the patient to touch the word “confirming” to manually advance to the next question. In another embodiment, an additional button labeled “Confirm” could be placed on the bottom of the screen to advance to the next question.

As a precautionary measure the interfaces shown in 610 and 620 contain options for the patient to go back a question by pressing the area of the display 630 or to repeat the current question by pressing the area 640. This allows maximum flexibility to assure that the correct answers are provided according to the patient's wishes. In an alternative embodiment, the bottom display could have other button options; for example, there could be a button to “Cancel” the history questionnaire, in which case the entire history is cancelled, or an “End” button, which might end the questionnaire at the current point and send it on to generate a report and alert the physician.

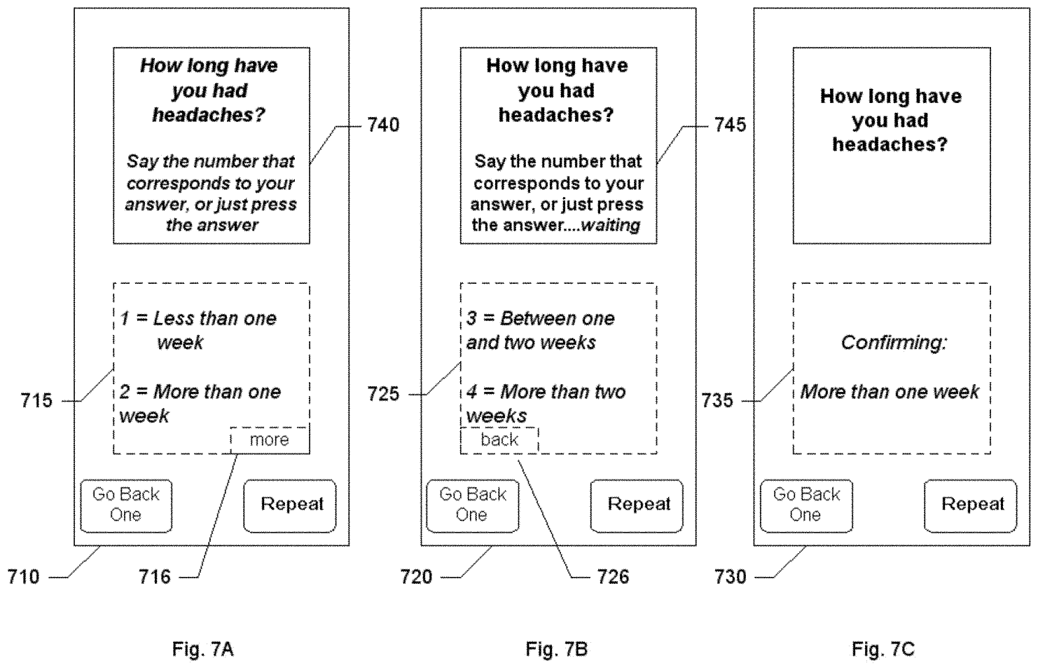

Questions that are concise might easily be displayed on a smartphone that might have a screen diameter size as small as 4 inches; however, if the questions become longer, they take up more of the display field. Moreover, should the required answer be among a long group of complex options, there would be no ability to show all the possible options on one page. With the present invention this issue may be solved, since the ability to speak questions makes it substantially easier to follow the available options. FIGS. 7A, 7B and 7C show three displays in which a more complex question is presented. Display 710 has a question presented in area 740, which, after being shown, is repeated by voice. Below that is section 715 in which a portion of the answers with their corresponding number are shown, and then repeated by voice. Once the display section 715 is shown and repeated by voice, a message appears on the bottom of the display section showing the word “more” 716. The display will automatically advance based on a timer, or the patient can touch the area 716 and manually advance the display. The next display 720 shows how the screen has changed. Area 740 adds the word “waiting” as shown in area 745, and area 715 updates to area 725 to show the remaining possible answers, which are repeated by voice. The patient can, at this point, say the numerical answer, or, if using a touch enabled smartphone, touch an answer in area 725, or choose to go back a screen to review the other options from screen 715. Using this approach an unlimited number of screens can be generated to handle more complex questions. Once the answer is chosen the display advances to the final screen 730 for a return voice to confirm the answer shown in area 735 “Confirming . . . more than one week” (which is shown as item #2 from area 715, Fig. A). In an alternative embodiment, the answers in screen 715 and 725 would not show up, but each answer would be entirely voiced while the single digit it represents is shown on the smartphone screen. For example, in display area 715 the number “1” would only appear and the words “Less than one week, press or say 1” would be spoken. After a predetermined period of time the number “2” would appear and the words “More than one week, press or say 2” would appear, and so forth until all four answers are given, and at the end of the answers the area 740 would change to reflect the same condition shown in area 745.

The aforementioned discussions related to presentation of the interface on the patient communication device, as presented in FIGS. 5A-B, 6A-B and 7A-B, is meant to provide one embodiment of the present invention. In an alternative embodiment, the entire process could be done entirely by voice. In this embodiment, the question, “How long have you had headaches” would be stated, followed by, “Press 1 if less than one week, Press 2 if more than one week, Press 3 if between one week and two weeks or Press 4 if more than two weeks.” The application would wait for an answer, and after a certain period of time, restate the question.

Other embodiments can exist and may include features such as a means for the patient to stop the questionnaire and terminate the program, a means to stop the questionnaire and start over, and a means for allowing the patient to go back multiple questions.

In yet another alternative embodiment, the patient can eliminate the voiced questions and responses and take the questionnaire by text only instructions. The entire voiced part of the question and answer section of the questionnaire, which starts after the complaint is provided, that appears on the patient device 350 through the medical history app 355 can be turned off such that the entire question and answer session can be completed in a more intimate setting through a text-only interaction, using the present invention. This embodiment provides a quiet interaction if the patient is in an area where other people are present. In this case, an introductory screen on the patient communication device 350 would show up prior to beginning the first question, and ask if the patient would like to eliminate the voiced sound and just use the text based questions. Implementation of this could be provided by either shutting down the voice/text processor 340, or simply suggesting to the patient to turn the speaker down on the patient communication device 350.

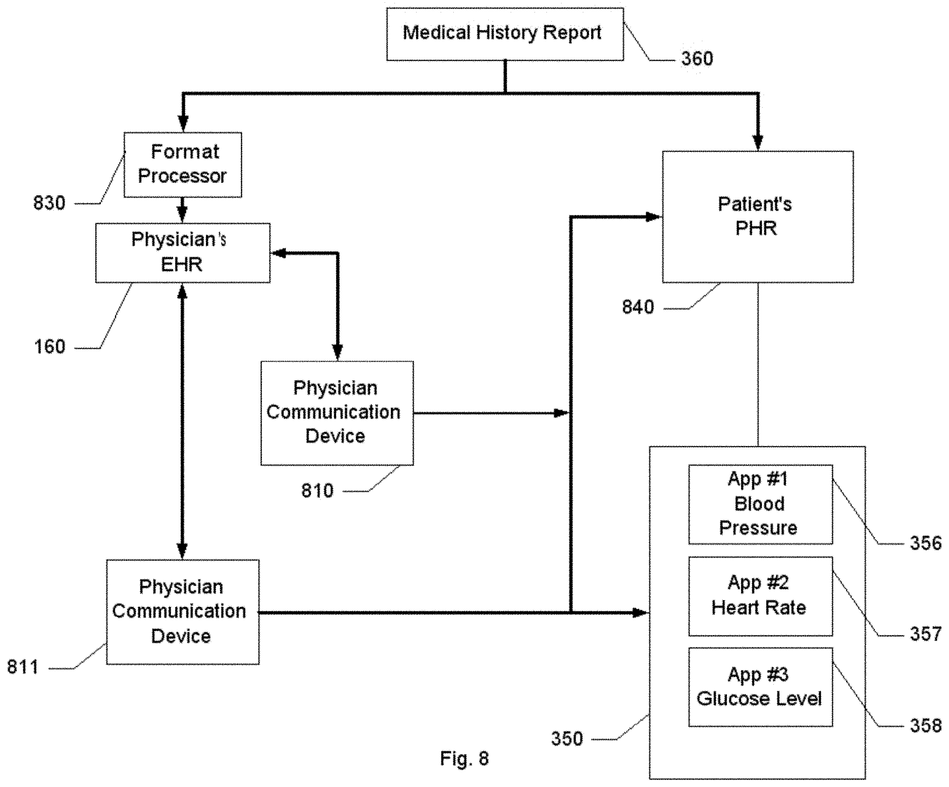

Once the entire questionnaire is completed the logic processor 320 prepares a text based report, formatted for quickly scanning by the physician, and sends the report on to the physician. FIG. 8 shows the system associated with the process of sending the medical history report 360 to the physician and subsequent disposition of the report. A copy of the medical history report 360 is sent by the logic processor 320 to the Physician's EHR 160 to be stored within the patient's EHR record. The report is then available for viewing by the physician along with other patient data held by the EHR. Once the EHR receives the report and has the report properly stored, the EHR automatically notifies the physician who is using a physician communication device 810, which, in the preferred embodiment of the invention, is a smartphone. However, as shown earlier in FIG. 2 the device according to the communication method and device described in FIG. 2, 230 could be either by voice using a smartphone, PC based phone or landline phone. In another embodiment the message could be sent via text message to a cellphone or smartphone. In another embodiment the message could be sent by email message.

Although some embodiments are constructed with the physician receiving the alert on the physician's communication device 810 from the physician's EHR. in alternative embodiments a second physician's communication device 811 monitored by a physician's assistant, nurse or nurse practitioner could receive the alert either simultaneously when the physician received the alert, or as an alternative to the physician receiving the alert.

Once the physician is contacted, the physician must read the medical history report 360. Depending on where the physician is and the device that is being used, the medical history report 360 is formatted specifically for optimum reading by the physician communication device 810 or 811 using a format processor 830 with a means for producing the medical history report 360 in various formats, such as text or voice based formats. As illustrative examples, suppose in one case the physician receiving the notification is a nurse who is in the physician's office, and can receive the medical history report 360 by way of an email with the report as an attachment from the EHR 160. The nurse could print out the attachment or read it on the computer screen, enter the EHR and review the patient's chart, and then call the patient to discuss the current complaint. In another case, however, the person receiving the notification could be the physician who is out playing golf and using a smartphone. If the report is not reformatted for the smartphone in some way, viewing it as a written document would be impossible, because the print would be too small. Therefore, the medical history report 360 would be reformatted to appear on the physician communication device 810 in such a way that allows the physician to read the report on a much smaller screen, then access the patient's record on the EHR 160, and then call the patient directly to discuss the issue at hand. The medical history report 360, formatted for the smartphone, would contain the same information as any other formatted version of the report.

In an alternative embodiment of the present invention the physician can receive the medical history report 360 in a completely voice enabled method. This allows, for example, for a physician to receive the report on a smartphone or smart watch with such a small display as to make the text formatting of the medical history to be sufficiently complex as to make it difficult to enact on a smartphone. When the text version of the history report is ready it can be processed by the voice/text processor 340 and sent to the physician's EHR as a fully voiced report.

Once the physician receives and understand the medical history report, the physician can review other information in the patient's EHR and then call the patient to discuss the patient's medical condition (FIG. 1, 190). At this point the physician and patient are in, at minimum, voice communication, and in the preferred embodiment, both the physician and patient are each on their respective smartphones.

Another aspect of the present invention is shown in FIG. 8. Smartphone technology allows the design and construction of “applications” or “apps,” which reside on the smartphone and can be used at any time by the owner of the smartphone. In most cases, apps are built such that they communicate in some way to a database that is usually held remotely, and accessed through the Internet protocol. The remote access database is sometimes referred to as the “Internet Cloud” or just “Cloud.” In the present disclosure the EHR and PHR would require apps to access their data through the Internet. A PHR allows a patient to collect and store any information related to the person's health. Generally, in the past, this is information that has been recorded elsewhere, such as data that has been put into a patient's record file in a physician's EHR. This might be, for example, blood pressure readings that are taken in a physician's office, put into the physician's EHR, and at the request of the patient, have the data transferred to the patient's PHR.

However, as opposed to generating the information elsewhere, another embodiment of the present invention allows for health related apps that can also run on a smartphone, and generate patient data that is unique and independent of the EHR 160, and data which can reside in the PHR 840. For example, there exists today apps the can monitor blood pressure 356 in real time, and store the data on the patient's smartphone 350 or in the patient's PHR 840, or on another third party cloud database. These apps operate by means of having an active sensor on the patient, recording clinical data and transferring the data to the smartphone. Although data transfer can occur by means of a wire, it is usually done by means of a short range bi-directional data transfer mechanism known by those familiar with the art as “Bluetooth” technology. Another example of a sensor based App would be using a sensor on the fingertip to record heart rate 357 and have the data deposited on the patient's PHR 840, retained on the smartphone 350, or sent to a third party database. Additionally, the data can be much more complex, as people familiar with the science of such devices understand that spectroscopic analysis is being miniaturized onto discrete components such that wearable devices can be enabled to read constituencies of chemical compounds, thereby allowing analysis of personal clinical data that heretofore would have to be measured in a laboratory setting. App #3 358 is an example of a direct glucose measurement from the smartphone using a connected device. In all cases the sensor-based patient data provides an ability to generate real time data about the patient, or provides most recent data depending on when the patient last ran the app.

Additional embodiments of external sensors are wearable sensors wherein a microprocessor based product takes the form of a bracelet or watch or other type of jewelry that is worn by a person, and has the ability to read health-related information about the patient. These wearable sensors can be simple devices that take a single piece of information, such as blood pressure, and communicate with the smartphone 350 to transfer the information to the EHR 160 or PHR 840. In other embodiments, however, the wearable sensors can be complex devices that connect to the smartphone 350 to connect to the EMR and PHR, or connect directly to the Internet to transfer data directly to the EHR and PHR without the necessity of going through the smartphone.

In addition to the sensor based apps above, smartphone apps are available that allow the patient to gather data about his or her medical condition and report the data to the PHR 840 without the presence of a sensor connected to the smartphone. There are also certain data that, at the present time, may not be able to be acquired by any sensor-type device that is attachable to a smartphone. In these cases data is gathered through an external means—going to clinical laboratory that will analyze the patient to get data—and the data is manually entered into the smartphone by the patient, where it can be integrated into the PHR 840 using a corresponding PHR App designed for that purpose.

At the time the physician calls a patient who has already taken a medical history, the physician will have at his disposal the Medical History Report 360 and access to patient data held in the physician's EHR 160; also, should the patient allow it, the physician can have access to the patient PHR 840, which may contain relevant data that was acquired by medical apps used with the smartphone and could be more recent data that the physician might want to see. Access would be granted optionally by the patient at the time of patient registration (FIG. 1, 120). Furthermore, should the patient allow it, the physician can have access to most recent data on the patient's smartphone 350, which has been inputted via a sensor or non-sensor based app described above, and not moved to the patients PHR 840. Or, the patient could run the sensor-based app while the physician is on the physician's smartphone, which has direct data communication access via an Internet or cellular connection to the patient's smartphone and both have the same application such that the physician could monitor the patient in real time by means of application software built specifically for the purpose of having the patient capture personal health related data in real time, then transfer said data to the physician's communication device such that the physician can recognize and review the data.

By having access to the communication avenues presented above between the patient and his physician, the physician would have all possible data available to make a valid and substantial clinical diagnosis of the patient from a remote location, in a manner that closely approximates the in-office examination, but in a much shorter period of time.

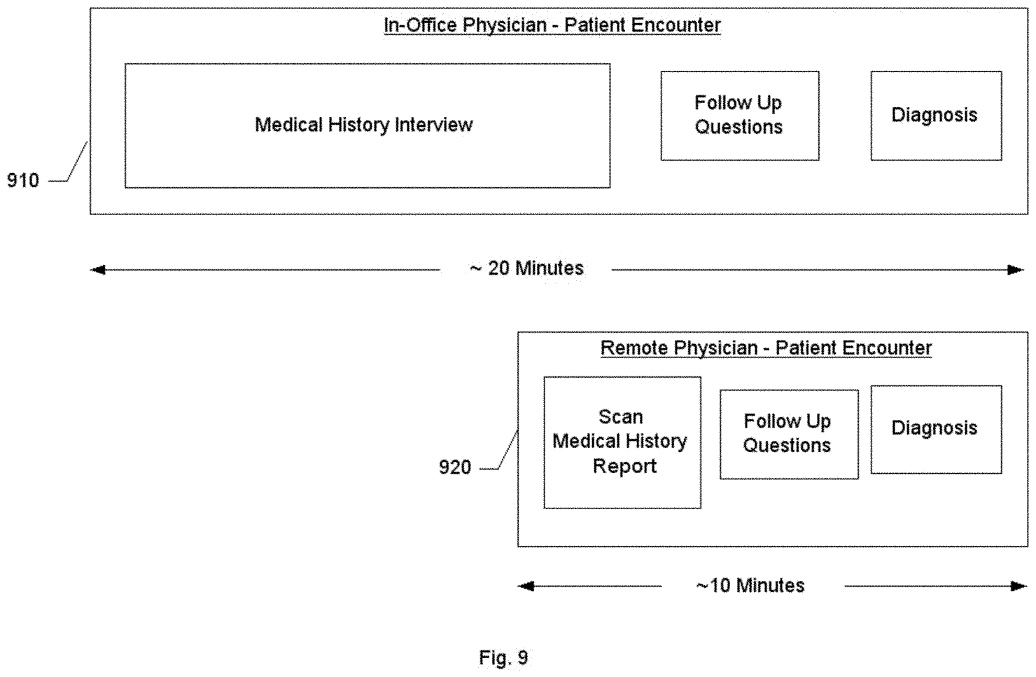

FIG. 9 is a graphical representation of the amount of time that is saved between a typical in-office visit and a remote visit using technology of the present invention. As previously mentioned and graphically depicted in area 910, a typical in-office visit takes about 20 minutes, and requires that the patient be onsite in the physician's office. The visit requires that a physician take a direct medical history interview with the patient, followed by a subjective question session to gather additional information from the patient, and lastly an assessment and diagnosis for next actions. With the aid of the present invention used in a remote setting, as shown in area 920, the session can be reduced to 10 minutes. The present invention eliminates direct contact by the physician to take a medical history, because patients initiate and complete their own medical history with an easy to use voice activated automated medical history to collect relevant medical history data for their physician. Patients perform vital necessary work on behalf of the physician, and actively, and substantially, contribute to their own care, without having to visit the physician's office.

Moreover, additional physician office time and resources are also saved by the present invention relative to an in-office visit. Resources which are eliminated include check in assistance at the front desk, guidance assistance to move the patient around the office, and check out assistance to release the patient. Additionally, if an in-office automated medical history questionnaire is administered, the patient has to be serviced by an office staff person, located at a computer, and in extreme, but often realized situations, the staff person has to be present at the computer to make sure the patient understands the process of using the automated medical history questionnaire, and will physically read the questions and press mouse and keyboard buttons to answer questions on behalf of the patient.

The technology of the present invention provides the ability for a physician to conduct a remote office visit of substantially the same quality of an in-office visit, in a small portion of the time and cost of an office visit, thereby increasing quality and productivity of the physician's office and the healthcare system in general.

The foregoing is illustrative of the present invention and is not to be construed as limiting thereof. Although exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed:

1. A method of providing diagnostic data for a chief diagnosis of a medical condition, comprising:
a) providing an Electronic Health Record (EHR) system, which includes medical records for patients of an individual physician;
b) providing an Internet-enabled, automated, branched logic, symptom-based medical history program (“Medical History Program”), which includes means for capturing text-based patient data associated with a chief medical complaint for a patient;
c) establishing a direct data link between the EHR system and the Medical History Program;
d) providing the patient with a computerized application program (“Medical History App”) to allow the patient to communicate with the Medical History Program; then
e) conducting a medical history interview initiated by the patient via the Medical History App, wherein the medical history interview comprises a plurality of questions answered by the patient, wherein at least 75 percent of the questions are closed-ended questions, the plurality of questions guided by the Medical History Program; and then
f) providing initial data for the patient to a physician based on the results of step (e), the initial subjective data being sufficient to enable the physician to render an initial diagnosis and begin collecting additional data to support the diagnosis.

2. The method defined in claim 1, wherein the Medical History App is provided on a remote communication device.

3. The method defined in claim 2, wherein step (e) is conducted via text capabilities.

4. The method defined in claim 2, wherein step (e) is conducted via voice recognition capabilities.

5. The method defined in claim 1, wherein the Medical History Program complies with HIPAA privacy guidelines.

6. The method defined in claim 1, wherein the at least 75 percent of closed-ended questions are yes-no questions.

7. The method defined in claim 1, further comprising (g) collecting objective data based on the initial diagnosis rendered in step (f).
